(19) United States
(12) Patent Application Publication
(10) Pub. No.: US 2009/0222005 A1
(43) Pub. Date: Sep. 3, 2009

(54) BONE CREST EXPANDER-COMPACTER AND ASSOCIATED TOOLS

(75) Inventor: Eduardo Anitua Aldecoa, Alava (ES)

Correspondence Address: BROWDY AND NEIMARK, P.L.L.C.;624 NINTH STREET, NW, SUITE 300, WASHINGTON, DC, 20001-5303, US

(73) Assignee: BTI,I+D, S.L., Vitoria (ES)

(21) Appl. No.: 12/282,423

(22) PCT Filed: Mar. 2, 2007
(86) PCT No.: PCT/IB2007/000571

§ 371 Date: Mar. 20, 2009

(30) Foreign Application Priority Data
Mar. 10, 2006 (ES) P 200600614

(51) Int. Cl. A61F 5/00

(52) U.S. Cl. 606/53

(57) ABSTRACT

Motor-driven hone crest expander-compacter (1) whose geometry and dimensions have been redesigned to optimal values that allow the expander-compacter (1) to bc used for bone expansion or compaction in rear areas of the patient's mouth. The expander-compacter (1) presents a threaded body (3) with a cylindrical threaded area (5) and a conical threaded area (6), where the cylindrical threaded area (5) presents a length smaller than two-thirds the length of the conical threaded area (6), in addition to the fact that the total length of the threaded body (3) is less than 12 mm. The invention also refers to a connector (7) connecting the expander-compacter (1) to a surgical motor, and to an initial burr (11), both of them equally optimised.

### TECHNICAL FIELD

The invention relates to a bone crest expander or tool used to expand or compact the bone crest of a patient prior to the installation of a dental implant.

### PRIOR ART

The prior art contains bone crest expanders or compacters, which are tools used to expand or compact the bone crest of a patient prior to the installation of a dental implant. Said expanders or compacters (hereinafter referred to as “expanders” or “expander-compacters”) can be driven manually or by a motor, where in the case of being motor-driven the expander is connected to a connector that can be operated by a surgical motor.

The use of motor-driven expanders provides some essential advantages during clinical application in comparison with the use of manual expanders. On the one hand, on inserting the expander with a micromotor instead of by hand, the control of the entry direction (or directionality) of the expander is much more precise. This is an important aspect bearing in mind that a bone is generally formed of parts with different density, and that the expander tends to be deflected when it moves from a bone part with a specific density to another bone part with a different density. On another hand, 10-15 N of force can be applied manually on the expander, whereas with a micromotor more than 60 N can be applied, thus enabling the insertion of the expanders into compact bones for the purpose of expanding or even breaking the bone intentionally.

Document WO-2004019807-A1, in name of the applicant, describes a set of motor-driven instruments that is used to prepare the maxillary bone for the installation of a dental implant, one of said instruments being a bone crest expander. Said document proposes a dental-implant installation procedure based on the use of a first tool or initial burr to break the cortical of the bone, a series of drills used to form a cavity in the bone, and a series of expanders used alternately with the drills to compact the walls of the bone cavity. Once the bone cavity presents dimensions and qualities suitable for housing the dental implant, the dental implant is inserted into the bone cavity, a period of time being necessary for its osseointegration to occur.

The aforementioned document describes other possible uses of the expanders, such as their usage for expanding a bone crest, thus enabling in this way the depth of the crest to be increased and the insertion of a dental implant of a greater length than that which the bone crest would have supported had it not been expanded.

In order to perform the expansion or compaction actions, the aforementioned document proposes expanders that comprise a head to receive one end of a connector, which on its other end is connected to a surgical motor. Furthermore, the expanders comprise a threaded body that presents a cylindrical threaded area, a conical threaded area and an apex or apical end. The conical threaded area fulfils the function of compacting the bone as the expander is inserted into the bone cavity, while the cylindrical threaded area allows the expander, as it is inserted into the bone cavity, to be threaded deeper and deeper taking hold in the bone and to increase its ability to advance.

In practice, it has been proven that the expanders used in the aforementioned document present certain drawbacks when used in rear areas of the patient's maxillary bone, where access is difficult and there is a very small interocclusal space in which to manoeuvre during surgery. The present invention aims to provide an expander design that is optimal for use in said rear areas of the patient's maxillary bone. The expander design is also useful for its application in patients with a small mouth opening.

### BRIEF DESCRIPTION OF THE INVENTION

It is an object of this invention to provide an expander-compacter whose geometry and dimensions have been redesigned to optimal values that allow the expander-compacter to be used for bone expansion or compaction in rear areas of the patient's mouth. Thus, the expander-compacter presents reduced dimensions in relation to other known expanders, having applied the dimension reduction in certain areas especially, for the purposes of not affecting the functioning of the expander-compacter. In this way, it can be considered that the inventive expander-compacter presents a minimum and optimised size for its application in areas that are difficult to access.

It is another object of this invention to provide a connector capable of being connected on one end to an expander and on its other end to a surgical motor, where the dimensions of the connector have also been redesigned and optimised so that the connector is minimal and can be used optimally in areas that are difficult to access.

It is another object of this invention to provide an initial burr, i.e., a tool that is used as an initial tool to break the cortical of the bone and drill the start of the bone cavity that will be drilled subsequently and widened in depth by expanders-compacters in conjunction with other drills. The initial burr according to the invention presents a geometry and dimensions maximised to an optimal size, which ensure that the initial burr does not only break the cortical of the bone and start the hole but that it also enters the bone throughout the depth of the required bone cavity. Using this initial burr, the expander-compacter does not have to drill and deepen, but rather it can enter the hole formed by the initial burr and only has to expand or widen it.

### BRIEF DESCRIPTION OF THE FIGURES

Details of the invention can be seen in the accompanying non-limiting drawings:

FIGS. 1 to 4 show a bottom view, a front view and a top view of four different embodiments of the expander-compacter according to the invention.

FIG. 5 shows a bottom view, a front view, a cross-sectional view and a top view of an embodiment of the connector according to the invention.

FIG. 6 shows a front view of an embodiment of the initial burr according to the invention.

### DETAILED DESCRIPTION OF THE INVENTION

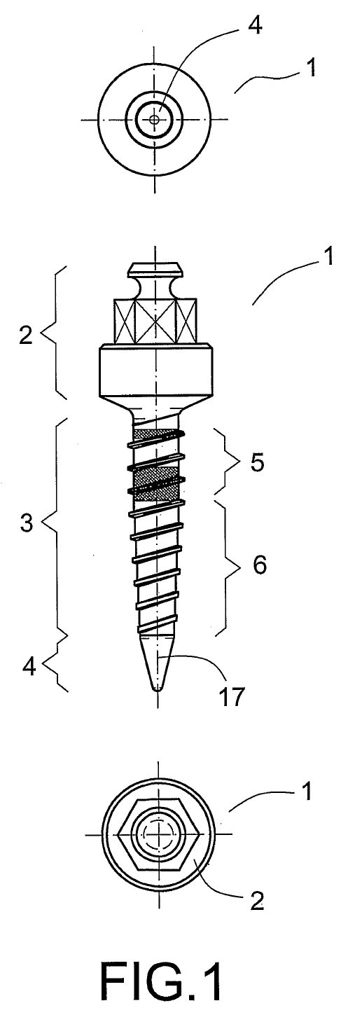

FIG. 1 shows a bottom view, a front view and a top view of a first embodiment of the bone crest expander-compacter according to the invention.

As can be seen in the figure, the expander-compacter (1) comprises a head (2), a threaded body (3) and an apical end (4). The head (2) acts as the connection area of a connector that is capable of being connected to a surgical motor to provide the motor-driven operation of the expander-compacter (1). For its part, the threaded body (3) performs the actual expansion or compaction. The threaded body (3), as in other known expanders, comprises a cylindrical threaded area (5) and a conical threaded area (6). According to the invention, the cylindrical threaded area (5) presents a length smaller than two-thirds the length of the conical threaded area (6), and the total length of the threaded area (3) is less than 12 mm—the “length” being understood as the dimension in the direction of the axis (17)-. These two geometrical and dimensional aspects provide an expander-compacter (1) of a minimum size that can enables access with correct directionality in areas that are difficult to access, and that performs and functions optimally.

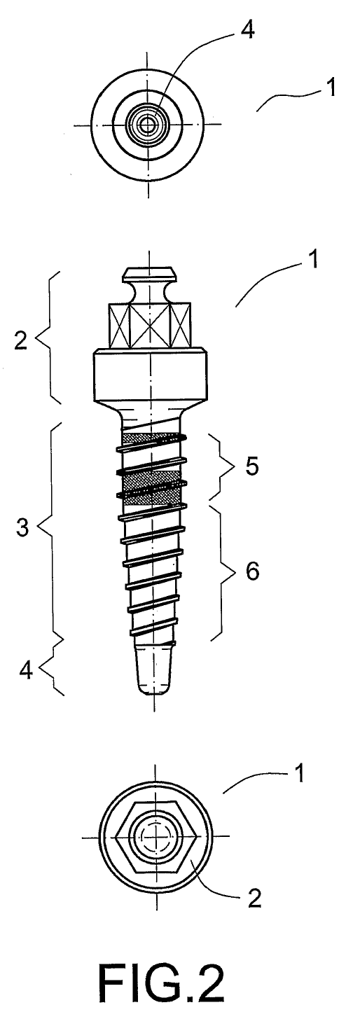

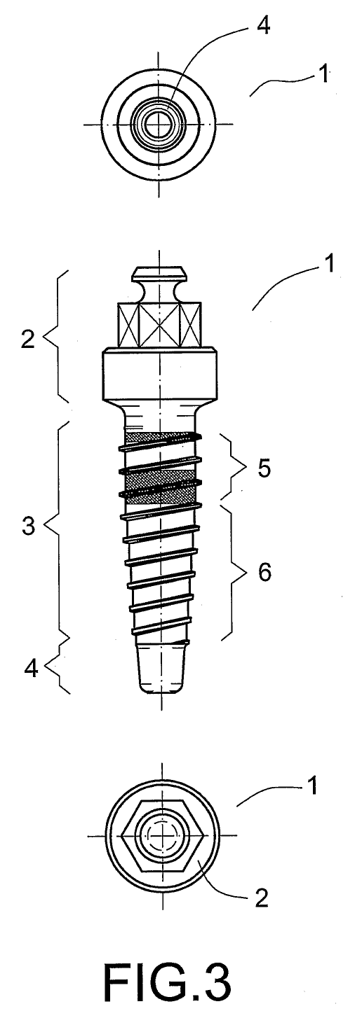

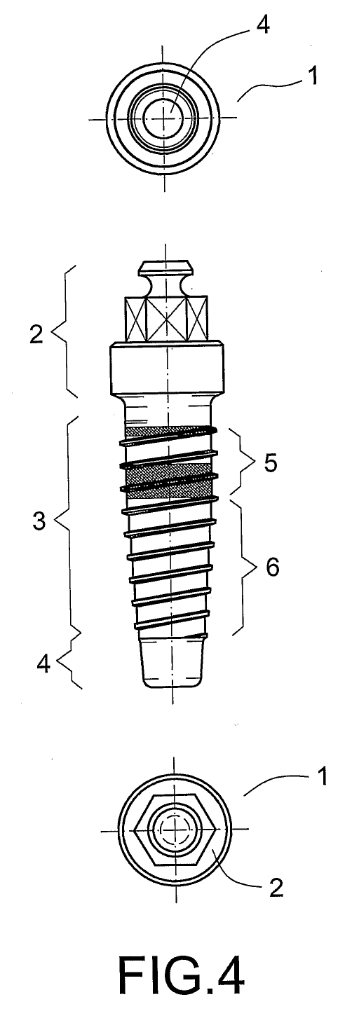

In an especially advantageous embodiment, the cylindrical threaded area (5) presents a length substantially between one third and one half of the length of the threaded area, and in this case, it is especially advantageous that the cylindrical threaded area (5) presents a length of between 2 and 4 mm and the conical threaded area (6) presents a length of between 6 and 9 mm. As a preferred solution the cylindrical threaded area (5) presents a length of substantially 3 mm and the conical threaded area (6) presents a length of substantially 7 mm. The embodiments of FIGS. 1 to 4 correspond to this especially advantageous embodiment, representing expanders-compacters (1) of different thicknesses. Specifically, in FIGS. 1 to 4 respectively, the external thread diameter of the cylindrical threaded area (5) is 2.5, 3.1, 3.6 and 4.3 mm.

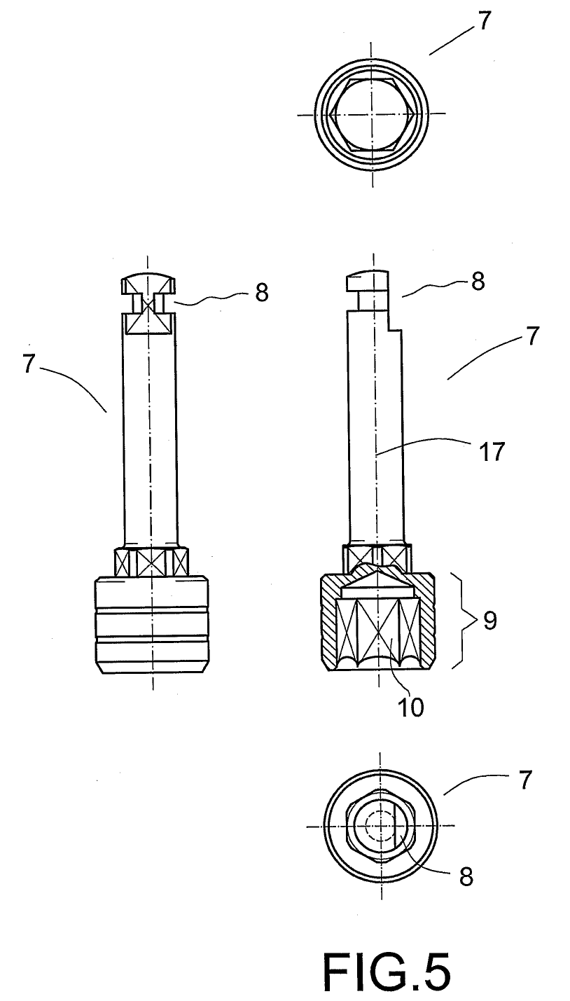

FIG. 5 shows a bottom view, a front view, a cross-sectional view and a top view of an embodiment of the inventive connector. As in other connectors known in the prior art, said connector (7) presents on one end a connection (8) to a surgical motor and on the other end it presents a head (9) that can be connected to the head (2) of an expander-compacter (1). For this reason, the head (9) presents an inner hollow (10) that receives at least part of the head (2) of the expander-compacter (1). The invention provides the connector (7) with specific dimensions that have been calculated and optimised so that the connector (1) is a sufficiently small size to be used in rear areas of the mouth, and at the same time performs correctly, unaffected by the reduction in dimensions. Thus, the head (9) presents a length of between 4 and 4.7 mm, and a diameter of between 4.7 and 5.3 mm. For its part, the inner hollow (10) presents a length of between 3.5 and 4 mm. The “diameter” is understood to be the dimension in a direction perpendicular to the axis (17). In an especially advantageous solution (represented in the figure), the head (9) presents a length of substantially 4.3 mm and a diameter of substantially 5 mm, and the inner hollow (10) presents a length of substantially 3.7 mm.

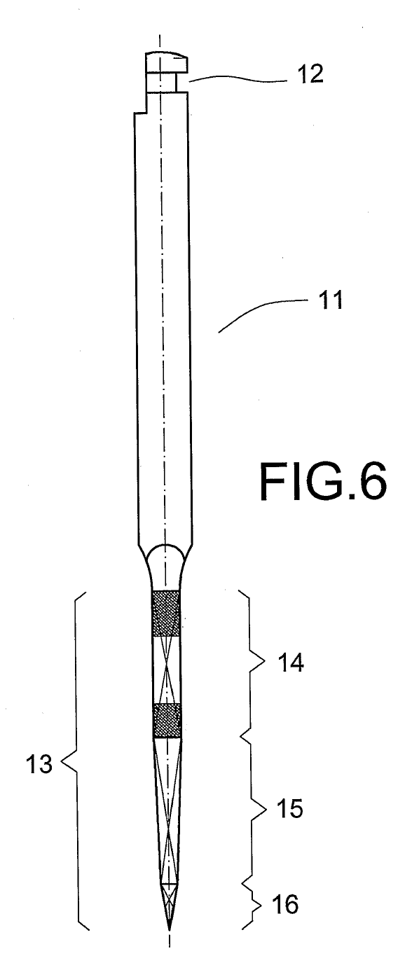

FIG. 6 shows a front view of an embodiment of the initial burr according to the invention. The initial burr (11) presents on one end a connection (12) to a surgical motor, and on the other end it presents a cutting tip (13), as other initial burrs designed by the applicant. The inventive initial burr (11) presents dimensions that are maximised and optimised to enable it to access rear areas of the mouth and also to allow it to start performing the bone cavity all along its length rather than only at the beginning of it. The purpose of said optimised dimensions is that the cutting tip (13) presents a length of between 9 and 20 mm, more preferably between 13 and 17 mm, and in an especially advantageous solution of substantially 15 mm (the solution represented in the figure). Additionally, in the embodiment of the figure the cutting tip (13) is divided into a cylindrical area (14), a conical area (15) and a conical apex (16), the conicity of the conical apex (16) preferably being greater that the conicity of the conical area (15), as has been represented. Being the initial burr (11) provided with said areas (14, 15, 16), it is especially advantageous that the cylindrical area (14) presents a length of substantially 6.5 mm, and the conical area (15) and the conical apex (16) present a total length of substantially 8.5 mm.

1. Bone crest expander-compacter (1), that comprises a head (2) for the connection of a connector capable of being connected to a surgical motor, a threaded body (3) and an apical end (4), where the threaded body (3) comprises a cylindrical threaded area (5) and a conical threaded area (6), characterised in that:
the length of the cylindrical threaded area (5) is less than two thirds the length of the conical threaded area (6),
the total length of the threaded body (3) is less than 12 mm.

2. Bone crest expander-compacter (1), in accordance with claim 1, characterised in that the length of the cylindrical threaded area (5) is substantially between one third and one half the length of the threaded area.

3. Bone crest expander-compacter (1), in accordance with claim 2, characterised in that the length of the cylindrical threaded area (5) is between 2 and 4 mm and the length of the conical threaded area (6) is between 6 and 9 mm.

4. Bone crest expander-compacter (1), in accordance with claim 3, characterised in that the length of the cylindrical threaded area (5) is substantially 3 mm and the length of the conical threaded area (6) is substantially 7 mm.

5. Connector (7), which on one end presents a connection (8) to a surgical motor and on the other presents a head (9) that can be connected to the head (2) of an expander-compacter (1), where said head (9) presents an inner hollow (10) that receives at least part of the head (2) of the expander-compacter (1), characterised in that:
the head (9) presents a length of between 4 and 4.7 mm and a diameter of between 4.7 and 5.3 mm,
the length of the inner hollow (10) is between 3.5 and 4 mm.

6. Connector (7), in accordance with claim 5, characterised in that the head (9) presents a length of substantially 4.3 mm and a diameter of substantially 5 mm, and the length of the inner hollow (10) is substantially 3.7 mm.

7. Initial burr (11), which on one end presents a connection (12) to a surgical motor, and on the other end presents a cutting tip (13), characterised in that the length of the cutting tip (13) is between 9 and 20 mm.

8. Initial burr (11), in accordance with claim 7, characterised in that the length of the cutting tip (13) is between 13 and 17 mm.

9. Initial burr (11), in accordance with claim 7, characterised in that the length of the cutting tip (13) is substantially 15 mm.

10. Initial burr (11), in accordance with claim 7, characterised in that the cutting tip (13) comprises a cylindrical area (14), a conical area (15) and a conical apex (16).

11. Initial burr (11), in accordance with claim 10, characterised in that the conicity of the conical apex (16) is greater than the conicity of the conical area (15).

12. Initial burr (11), in accordance with claims 9 and 10, characterised in that the cylindrical area (14) presents a length of substantially 6.5 mm, and the conical area (15) and the conical apex (16) present a total length of substantially 8.5 mm.
